SEQUENCE LISTING (6 sequences)

SEQ ID NO: 1 - DNA, 2940 bp, Arabidobsis Thaliana
  CDS (336)...(2492)
gttttttcttctgaagagtgatatatattctacctttctctggttaaagaaactccctgaatccaccggttatgtcttgaccggctttaagcctataaactgatgccctaagacacctttttaggtttctcaataattctccgcatctatcttttcttctccacaagtaagggaaccagaaaaccagggaagaatccgagcaagctagggtttcattgtgtgcacaaaatgggatatacaggcagaagaaaatcgagataaatcaactaaatgatttggataatcatcttgaagatttgaaggatttcgagactaagtccggcgcagaagtcaccatggagaatcctttagaagaagagcttcaagatcctaatcagcgtcccaacaaaaagaagcgttaccaccgtcacacacaacgccagattcaagagctagagtcgttcttcaaggaatgtcctcatccagacgataagcaaagaaaggagctgagtcgcgagctaagcttagaacctcttcaagtcaagttctggttccaaaacaaacgcactcaaatgaaggcacaacatgagaggcacgagaaccagatactgaagtcagaaaatgacaagctccgagcagagaacaataggtacaaggatgctctaagcaacgcaacatgcccaaactgtggtggtccggcagctataggagaaatgtccttcgacgaacagcatttaaggatcgaaaatgctcgtttacgcgaagagattgacagaatctctgccatagctgctaaatacgtagggaagcctttaatggctaattcctcttctttccctcagctctcttcttcacaccacattccctcgcgctcgcttgatcttgaagttgggaactttgggaacaataacaatagccacactggtttcgttggggaaatgtttggaagcagcgacattttgaggtcggtttcgataccttctgaggctgataagcctatgattgttgagttagctgttgcagcaatggaagagcttgtgagaatggctcaaactggtgatcccttatgggtttcaagcgataattctgttgagattctcaatgaagaagagtattttaggacgtttcctagaggaattggaccgaaacctatcggtttgagatcagaagcttcaagagagtctactgttgttatcatgaatcatatcaatctcattgagattctaatggatgtgaatcaatggtctagtgtgttctgcgggattgtatcaagagcattgactctagaagttctctcaactggcgtacgagggaactacaatggggcattgcaagtgatgacagcagagttccaagtcccatcgccgcttgtccctactcgtgagaactactttgtaaggtactgtaaacagcacagtgacggtatttgggcggttgtggatgtctctttggacagcctaagaccaagtccgatcactagaagcagaagaagaccctctggttgtctgattcaagaattgcagaatggttactccaaggtgacatgggtagagcatattgaggtggatgatagatcggttcacaacatgtataaaccgttggttaataccggtttagctttcggtgcaaaacgttgggtggctacacttgaccgccaatgtgagcggctcgccagttccatggccagcaacattccggcttgtgatctttccgtgataacgagtcctgaggggagaaagagcatgctgaaactagcggagagaatggtgatgagcttctgtaccggagtcggcgcgtcaaccgccgatgcctggactacattgtcgaccacaggatccgacgacgttcgggtcatgacccgaaagagcatggatgatccgggaagacctccaggcatcgttctcagcgccgctacttctttctggatccctgtagctccaaaacgagtgttcgattttctcagagatgaaaactcaagaagcgagtgggatatactttccaatggaggcttggttcaagaaatggctcatatcgcaaatggtcgtgatcctgggaatagtgtctccttgcttcgagtcaatagtgggaactcagggcagagcaacatgttgatcttacaagaaagttgtacggacgcatcagggtcctatgtgatatacgcaccagttgatataatagctatgaacgttgtcctgagtggtggtgatccggattatgtcgctttgttaccatccggattcgctattttgccggatggctctgctagaggaggaggaggtagtgctaatgccagtgctggagccggagttgaaggaggaggagaggggaataatcttgaagtggttactactactgggagttgtggcggttcactactcacagttgcgtttcagatacttgttgactctgttcctaccgctaaactctctctcggttcagttgctacagtcaatagtctgatcaaatgcactgtcgagcggattaaagccgctctggcctgcgacggagcctaatcgatgttttcggaaggtaagagtgaaaggggaggtttagggagtttatgataatgtttgtgttcttttggtttttaaagtcttttgagattctccaaaggaagtcaagaacgctcctttttgcgtttaatctcatttccgcgtttgttagcggacgggccaaagaaagaggcttgagaaagaaaaggtaaagaggttcgggtattgacttctgctggaaccaaaaaaaaaggaatcgggtttgttgtgtttcggcggtttagcattttgcgttttctttgttattatttatcattgactagtgaacagtttagcgttctgcttttcgcgtctactgtgaaactccttgttattaagccactctagtggtactgtcattatatattatgaatctatgaaactgtgtttattagtttgtttctttaatccaaacttgagattctcttct

SEQ ID NO: 2 - protein, 718 aa, Arabidobsis Thaliana
MENPLEEELQDPNQRPNKKKRYHRHTQRQIQELESFFKECPHPDDKQRKELSRELSLEPLQVKFWFQNKRTQMKAQHERHENQILKSENDKLRAENNRYKDALSNATCPNCGGPAAIGEMSFDEQHLRIENARLREEIDRISAIAAKYVGKPLMANSSSFPQLSSSHHIPSRSLDLEVGNFGNNNNSHTGFVGEMFGSSDILRSVSIPSEADKPMIVELAVAAMEELVRMAQTGDPLWVSSDNSVEILNEEEYFRTFPRGIGPKPIGLRSEASRESTVVIMNHINLIEILMDVNQWSSVFCGIVSRALTLEVLSTGVRGNYNGALQVMTAEFQVPSPLVPTRENYFVRYCKQHSDGIWAVVDVSLDSLRPSPITRSRRRPSGCLIQELQNGYSKVTWVEHIEVDDRSVHNMYKPLVNTGLAFGAKRWVATLDRQCERLASSMASNIPACDLSVITSPEGRKSMLKLAERMVMSFCTGVGASTADAWTTLSTTGSDDVRVMTRKSMDDPGRPPGIVLSAATSFWIPVAPKRVFDFLRDENSRSEWDILSNGGLVQEMAHIANGRDPGNSVSLLRVNSGNSGQSNMLILQESCTDASGSYVIYAPVDIIAMNVVLSGGDPDYVALLPSGFAILPDGSARGGGGSANASAGAGVEGGGEGNNLEVVTTTGSCGGSLLTVAFQILVDSVPTAKLSLGSVATVNSLIKCTVERIKAALACDGA

SEQ ID NO: 3 - DNA, 200 bp, Arabidobsis Thaliana
cttgaagatttgaaggaaaatccaagagcttcaaaaactccaaaaattgataggcatccatcatcatcatgtatcatccaaacatgttcgaatctcatcatcatatgttcgatatgacgccgaaaaactccgaaaacgatttgggtatcaccgggagccacgaagaggatttcgagactaagtccggcgcagaagtcacc

SEQ ID NO: 4 - DNA, 551 bp, Arabidobsis Thaliana
aagcagcgacattttgaggtcggtttcgataccttctgaggctgataagcctatgattgttgagttagctgttgcagcaatggaagagcttgtgagaatggctcaaactggtgatcccttatgggtttcaagcgataattctgttgagattctcaatgaagaagagtattttaggacgtttcctagaggaattggaccgaaacctatcggtttgagatcagaagcttcaagagagtctactgttgttatcatgaatcatatcaatctcattgagattctaatggatgtgaatcaatggtctagtgtgttctgcgggattgtatcaagagcattgactctagaagttctctcaactggcgtacgagggaactacaatggggcattgcaagtgatgacagcagagttccaagtcccatcgccgcttgtccctactcgtgagaactactttgtaaggtactgtaaacagcacagtgacggtatttgggcggttgtggatgtctctttggacagcctaagaccaagtccgatcactagaagcagaagaagaccct

SEQ ID NO: 5 - DNA, 25 bp, Artificial Sequence
  Artificial PCR Primer
aaaaagcttagtctcgaaatccttc

SEQ ID NO: 6 - DNA, 26 bp, Artificial Sequence
  Artificial PCR Primer
tttaagcttaaccggtggattcaggg

(19) United States
(12) Patent Application Publication
(10) Pub. No.: US 2002/0056153 A1
(43) Pub. Date: May 9, 2002

(54) Epidermal specific regulatory sequence

(76) Inventors: Martin Yanofsky, San Diego, CA (US); Allen Sessions, Encinitas, CA (US); Detlef Weigel, Solana Beach, CA (US)

Correspondence Address: KNOBBE MARTENS OLSON & BEAR LLP, 620 NEWPORT CENTER DRIVE, SIXTEENTH FLOOR, NEWPORT BEACH, CA, 92660, US

(21) Appl. No.: 09/823,635

(22) Filed: Mar. 30, 2001

Related U.S. Application Data
(60) Provisional application No. 60/193,733, filed on Mar. 30, 2000.

(51) Int. Cl. C12N 15/82; A01H 5/00; C12N 15/29

(52) U.S. Cl. 800/287; 800/302; 536/23.6; 536/24.1

(57) ABSTRACT

A novel, epidermal-specific regulatory sequence is provided which has been isolated from the 5′ end of a plant ML1 gene. This tissue-specific regulatory sequence, operably associated with a nucleic acid sequence expressing a product of interest, initiates and regulates the transcription of the nucleic acid sequence in the L1 layer of a plant epidermis in meristems and young primordia.

### RELATED APPLICATIONS

This application claims priority to U.S. Provisional Patent Application No. 60/193,733, filed on Mar. 30, 2000 entitled INTRACELLULAR MOVEMENT OF A TRANSCRIPTION FACTOR IN ARABIDOPSIS FLORAL MERISTEMS AND THE ARABIDOPSIS THALIANA MERISTEM LAYER 1 REGULATORY SEQUENCE SPECIFIES EPIDERMAL EXPRESSION IN MERISTEMS AND YOUNG PRIMORDIA.

### BACKGROUND OF THE INVENTION

1. Field of the Invention

The present invention relates generally to plant genetic engineering. In addition, the invention relates to the identification of regulatory sequences involved in epidermis-specific expression in plant cells. More particularly, the invention relates to a Meristem Layer 1-specific regulatory sequence capable of directing epidermal expression of heterologous genes in meristems and young primordia.

2. Description of the Related Art

Shoot apical meristems (SAMs) of higher plants are stratified into cell layers. Hanstein was the first to recognize that meristems have an epidermal layer, or L1, that covers distinct internal cell layers, or L2 and L3. Hanstein, J Festschrift der Niederrrheinhischen Gesellschaftfr Natur-u. Heilkunde, 109-134 (1868). The L1 is a single layer of cells, whereas the L2 and L3 can each comprise more than one cell layer. Restrictions in the plane of cell division within the outer layers L1 and L2 lead to the generation of two or three clonally distinct populations of cells in the SAM and its derivatives, as first shown in detail by Satina and colleagues. Satina, S. et al. Am. J Bot. 44, 311-317 (1940). In general, the L1 forms the epidermis and in some cases internal tissue at the margins of organs, the L2 gives rise to mesophyll and subepidermal layers of organs as well as to the germ line, and the L3 gives rise to the ground tissue and vasculature. The significance of SAM stratification is unclear, and it is not known whether it is causative of pattern or reflective of steric constraints. Further, although the layers behave largely independently in their cell division patterns, they communicate during organ formation, as many studies have shown (reviewed in Szymkowiak, E. J. and Sussex, I. M., Ann. Rev. Plant Mol. Biol. 47, 351-376 (1996)).

Recently, several genes that are expressed in a meristem layer-specific manner have been described. For the L1, these include Arabidopsis thaliana MERISTEM LAYER 1 (ATML1) and LIPID TRANSFER PROTEIN 1 from Arabidopsis (See, Lu et al., Plant Cell 8, 2155-2168 (1996); Thoma et al., Arabidopsis. Plant Physiol. 105, 35-45 (1994)). For the L2/L3 these include KNOTTED1 from maize, A3 from tobacco, CENTRORADIALIS from snapdragon, and CLAVATA1 from Arabidopsis (Jackson et al., Development 120, 405-413 (1994); Kelley and Meeks-Wagner, Plant J., 8, 147-153 (1995); Bradley et al., Antirrhinum. Nature 379, 791-797 (1996); Clark et al., Cell 89, 575-585 (1997)). The regulatory sequences responsible for layer-specific expression in meristems have not been identified for many of these genes. For CLV1; H. Schoof et al., Cell 100, 635-44 (2000) used CLV1 regulatory sequences to drive a chimeric transcription factor that in turn activated GUS.

The ATML1 locus encodes a homeodomain protein that is transcribed at high levels in the epidermis of developing embryos, and the L1 of shoot and floral meristems (Lu et al., Plant Cell 8, 2155-2168 (1996). Expression begins in the apical cell of the one cell embryo, becomes restricted to the protoderm at the 16-cell stage of embryogenesis, and later is expressed in the L1 of shoot and floral, but not root, meristems. Id.

To date, the regulatory sequences responsible for layer-specific expression in meristems have not been fully identified. Accordingly, there is a need for identifying epidermal specific regulatory sequences to drive epidermis-specific expression of heterologous genes in shoot, floral and root meristems.

### SUMMARY OF THE INVENTION

Embodiments of the invention provide an epidermal tissue-specific regulatory sequence that is useful for expressing a gene in the epidermal layer of a plant. In one aspect of the invention, an isolated epidermal specific regulatory nucleic acid is provided wherein operably associating the regulatory nucleic acid with a heterologous nucleic acid results in epidermal specific expression of the heterologous nucleic acid. Advantageously, the epidermal specific regulatory nucleic acid is derived from an ATML1 gene. In some aspects of the invention, the epidermal specific regulatory nucleic acid is a ML1 epidermal specific regulatory nucleic acid. The ML1 epidermal specific regulatory nucleic acid may include the sequence of SEQ ID NO: 3, or functional fragments thereof. Preferably, the heterologous nucleic acid encodes for a gene which confers resistance to disease or pests such as the Bacillus thuringiensis toxin.

In another aspect of the invention, there is provided a transgenic plant cell which includes an epidermal specific regulatory nucleic acid operably associated with a heterologous nucleic acid, wherein a plant derived from the plant cell specifically expresses the heterologous nucleic acid in the epidermis. The transgenic plant cell may be a monocotyledonous or dicototyledonous plant cell. In some aspects of the invention, the transgenic plant cell is a tobacco or Arabidopsis plant cell.

The epidermal specific regulatory nucleic acid may be derived from an ATML1 gene. In one aspect of the invention, the epidermal specific regulatory nucleic acid is an ML1 epidermal specific regulatory nucleic acid. The ML1 epidermal specific regulatory nucleic acid may include the sequence of SEQ ID NO: 3 or functional fragments thereof. The heterologous nucleic acid may encode a Bacillus thuringiensis toxin. Optionally, the transgenic plant cell further includes a selectable marker.

In one aspect of the invention, there is provided a transgenic plant including an epidermal specific regulatory nucleic acid operably associated with a heterologous nucleic acid, wherein the heterologous nucleic acid is specifically expressed in the epidermal layer of the plant. The plant may be a monocotyledonous or a dicotyledonous plant. In one aspect of the invention, the plant is Arabidopsis thaliana. In another aspect of the invention, the plant is a tobacco plant. Optionally, the transgenic plant includes a selectable marker.

Advantageously, the transgenic plant includes an epidermal specific regulatory nucleic acid derived from an ATML1 gene. The epidermal specific regulatory nucleic acid may be a ML1 epidermal specific regulatory nucleic acid. The ML1 epidermal specific regulatory nucleic acid may include the sequence of SEQ ID NO: 3, or functional fragments thereof. Advantageously, the heterologous nucleic acid encodes a Bacillus thuringiensis toxin.

An expression vector including an epidermal specific regulatory nucleic acid operably associated with a heterologous nucleic acid is likewise provided. The expression vector may include a retroviral vector, a Ti plasmid, or a Cauliflower mosaic virus (CaMV). The epidermal specific regulatory nucleic acid is advantageously derived from an ATML1 gene. In some aspects of the invention, the epidermal specific regulatory nucleic acid is a ML1 epidermal specific regulatory nucleic acid. The ML1 epidermal specific regulatory nucleic acid may include the sequence of SEQ ID NO: 3, or functional fragments thereof. Preferably, the heterlogous nucleic acid encodes a Bacillus thuringiensis toxin.

A method of specifically expressing a heterologous nucleic acid in the epidermis of a plant is described. The method includes transforming a plant with a nucleic acid construct comprising an epidermal specific regulatory nucleic acid operably associated with the heterologous nucleic acid and selecting for plants that exhibit epidermal specific expression of said heterologous nucleic acid. The plant to be transformed may be an Arabidopsis thaliana plant or a tobacco plant. Advantageously, the heterologous nucleic acid encodes a Bacillus thuringiensis toxin.

### BRIEF DESCRIPTION OF THE DRAWING

FIG. 1 is a diagram of the 5′ region of ATML1 and the map of fragments fused to GUS. Exons are indicated by open boxes, the putative transcription start site is labeled with an arrow, and the putative initiation ATG in the third exon is indicated. PAS76 and pAS85 are genomic subclones. Sites of primer 83 and primer 87 are indicated. B, BamHI; H, HindIII; X, Xba1.

### DETAILED DESCRIPTION OF THE PREFERRED EMBODIMENT

The epidermis of a plant is the first line of defense for a plant against pests and diseases. The current practice for genetic engineering of traits into a plant employs constitutive regulatory sequences which express genes responsible for the traits in every cell of the plant. Such ubiquitous expression of genes has inherent drawbacks, such as expressing the gene in tissues throughout the plant. Thus, it would be advantageous to provide a system whereby the gene of interest is only expressed, for example, the epidermal layer of the plant. This would provide a mechanism for specifically expressing foreign toxins providing pest or disease resistance only in the skin layer, which is a plant's first line of defense, rather than throughout the plant. Embodiments of the invention described herein provide such a system.

Embodiments of the invention provide an epidermal tissue-specific regulatory sequence that is useful for expressing a gene in the epidermal layer of a plant. Additionally, methods of engineering traits into the epidermis of plants using the layer-specific regulatory sequence are contemplated. In accordance with an aspect of the invention, a nucleic acid construct is described which allows a plant phenotype to be modified based on expression of the desired gene in the L1 layer of the plant epidermis. The nucleic acid construct advantageously includes a gene of interest which provides for the modification in phenotype, positioned downstream from, and under the transcriptional initiation regulation of, an epidermal-specific regulatory sequence (ESRS).

Epidermal-specific regulatory sequences can be utilized for expression of genes of interest in the epidermis of a plant. As used herein, the term “epidermal specific regulatory sequence” includes nucleic acid sequences involved in the regulation of gene expression specifically in the epidermis of plants. Linking an ESRS with a heterologous gene of interest leads to expression of the heterologous gene specifically in the epidermal layer of the plant. Such genes of interest include genes that protect against disease, genes conferring protection against various pests, genes involved in pigmentation, and genes involved in wax and oil composition, or other traits.

In one embodiment, the ESRS is derived from a Meristem Layer I-specific regulatory sequence (SEQ ID NO: 3). The Meristem Layer 1 (ML1) regulatory sequence is useful for specifically expressing a gene of interest in the epidermal layer of a plant. In one embodiment, the invention includes a nucleic acid construct including a non-coding regulatory sequence isolated from the 5′ end of a plant Meristem Layer 1 (ML1) gene, wherein the non-coding regulatory sequence is operably associated with a nucleic acid sequence expressing a product selected from a protein of interest or antisense RNA and wherein the nucleic acid sequence is heterologous to the non-coding sequence. The construct advantageously includes a transcriptional and translational initiation region and a transcriptional and translational termination region functional in plants. In preparing the construct, the various component nucleic acid sequences are manipulated, so as to provide for nucleic acid sequences in the proper orientation and proper reading frame.

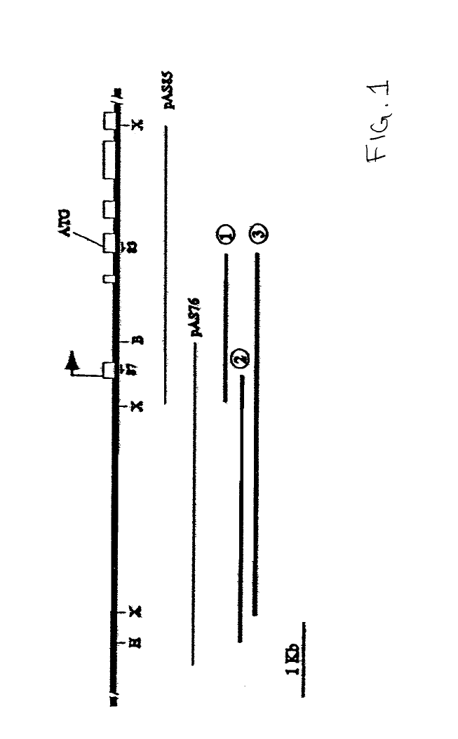

The ML1 ESRS is located in the non-coding region of the ATML1 gene and was found to provide strong expression in the epidermis of meristems. The 5′ region of ATML1 is shown as a diagram in FIG. 1. The transcription start is tentatively identified as the 5′ end of the longest cDNA derived from ATML1 Qu et al., 1996) (SEQ ID NO: 1). The alignment of FIG. 1 shows that transcription starts at least 1.8 kb upstream of the predicted first ATG, which lies in the third exon, as described in the Examples herein.

Placing a heterologous nucleic acid sequence expressing a product of interest under the control of an ESRS, such as the ML1 ESRS means positioning the heterologous nucleic acid sequence such that expression is controlled by the regulatory sequence. In general, regulatory sequences are positioned upstream of the genes that they control and typically include regulatory sequences in addition to other regulatory elements such as enhancers and repressors. Thus, in the construction of the regulatory sequence/gene combinations, the regulatory sequence is preferably positioned upstream of the gene and at a distance from the transcription start site that approximates the distance between the regulatory sequence and the gene it controls in its natural setting. As is known in the art, some variation in this distance can be tolerated without loss of regulatory sequence function. Similarly, the preferred positioning of a regulatory element with respect to a gene placed under its control reflects its natural position relative to the structural gene it naturally regulates. Again, as is known in the art, some variation in this distance can be accommodated.

As discussed herein, an ESRS typically includes a promoter and other regulatory elements, such as an enhancer or repressor. Of course, it should be realized that the regulatory elements described herein which control epidermal specific expression of a gene of interest can also be linked to a heterologous promoter and functional fragments thereof in order to provide the promoter with epidermal-specific expression characteristics. Thus, an ESRS should not be construed to be limited to only those sequences that include promoters.

Accordingly, as used herein, the term “functional fragments” of an ESRS refers to fragments of a the ESRS that when linked to a heterologous nucleic acid, or promoter/heterologous nucleic acid combination, alter the transcriptional activity of the heterologous nucleic acid. For example, a functional fragment can be an enhancer which enhances the transcriptional activity of an endogenous or exogenous promoter. Alternatively, the functional fragment can be a repressor, which represses the transcriptional activity of the endogenous or exogenous promoter.

Functional fragments of heterologous promoters can be generated in any number of ways known to one of skill in the art. For example, overlapping fragments of the regulatory sequence of a promoter can be synthesized and then evaluated for functionality by splicing the synthetic fragments into an expression system and identifying differential expression. Alternatively, functional fragments can be ascertained via foot print analysis, a technique commonly known by one of skill in the art whereby protein binding sites in DNA are mapped. See Galas, D. and Schmitz, A., Nucleic Acid Res. 5:3161 (1978).

ESRS function and tissue layer-specific expression of a gene under the regulatory control of an ESRS can be tested at the transcriptional stage using DNA/RNA and RNA/RNA hybridization assays (in situ hybridization) and at the translational stage using specific functional assays for the protein synthesized (for example, by enzymatic activity or by immunoassay of the protein).

As used herein, the term “nucleic acid sequence” refers to a polymer of deoxyribonucleotides or ribonucleotides, in the form of a separate fragment or as a component of a larger construct. Nucleic acids expressing the products of interest can be assembled from cDNA fragments or from oligonucleotides which provide a synthetic gene which is capable of being expressed in a recombinant transcriptional unit. Polynucleotide or nucleic acid sequences include DNA, RNA, and cDNA sequences.

Nucleic acid sequences can be obtained by several methods. For example, the DNA can be isolated using hybridization procedures which are well known in the art. These include, but are not limited to: 1) hybridization of probes to genomic or cDNA libraries to detect shared nucleotide sequences; 2) antibody screening of expression libraries to detect shared structural features; and 3) synthesis by the polymerase chain reaction (PCR). Sequences for specific genes can also be found in GenBank, National Institutes of Health computer database.

The phrase “nucleic acid sequence expressing a product of interest” refers to a structural gene which expresses a product selected from a protein of interest or antisense RNA. The term “structural gene” excludes the non-coding regulatory sequence which drives transcription. The structural gene may be derived in whole or in part from any source known to the art, including a plant, a fungus, an animal, a bacterial genome or episome, eukaryotic, nuclear or plasmid DNA, cDNA, viral DNA or chemically synthesized DNA. A structural gene may contain one or more modifications in either the coding or the untranslated regions which could affect the biological activity or the chemical structure of the expression product, the rate of expression or the manner of expression control. Such modifications include, but are not limited to, mutations, insertions, deletions, and substitutions of one or more nucleotides. The structural gene may constitute an uninterrupted coding sequence or it may include one or more introns, bound by the appropriate splice junctions. The structural gene may also encode a fusion protein. It is contemplated that the introduction into plant tissue of nucleic acid constructs of the invention will include constructions wherein the structural gene and its regulatory sequence, e.g., ML1 regulatory sequence, are each derived from different plant species.

The term “heterologous nucleic acid sequence” as used herein refers to at least one structural gene which is operably associated with the epidermal specific regulatory sequence of the invention. The heterologous nucleic acid sequence originates in a foreign species, or in the same species if substantially modified from its original form. For example, the term “heterologous nucleic acid sequence” includes a nucleic acid originating in the same species, where such sequence is operably linked to the same species, where such sequence is operably linked to a regulatory sequence that differs from the natural or wild-type epidermal specific regulatory sequence (e.g., ML1 regulatory sequence).

The term “operably associated” refers to functional linkage between a regulatory sequence and a nucleic acid sequence. The operably linked regulatory sequence controls the expression of the nucleic acid sequence.

Examples of heterologous structural genes that may linked to an ESRS so as to be expressed in the epidermal layer of a plant include the Bacillus thuringiensis toxin gene which provides pest/pathogen protection. The sequence for the Bacillus thuringiensis toxin gene has been well-characterized. Genes encoding the crystal proteins have been identified and cloned from several varieties of Bacillus thuringiensis, including, for example, the protoxin genes from kurastaki strains HD-1 and HD-1-Dipel (Whiteley et al. (1982) in Molecular Cloning and Gene Regulation in Bacilli, Ganesan et al. (ed) pp. 131-144; Schnepf and Whiteley (1981) Proc. Natl. Acad. Sci. USA 78:2893-2897 and U.S. Pat. Nos. 4,448,885 and 4,467,036). Similarly, the nucleotide sequence of the Bacillus thuringiensis toxin protein have been reported. See, e.g., Wong et al., J Biol. Chem. 258:1960-1967 (1983); and Schnepf and Whiteley, J Biol,. Chem 260:6264-6272 (1985).

Other genes that would be advantageously overexpressed in the epidermis include genes controlling trichomes, such as CPC (Wada, et al. Okada, Science 277, 1113-6 (1997) or GL1 (Larkin, et al., Plant Cell 6, 1065-1076 (1994); or genes controlling epidermal waxes such as CER genes (Hannoufa, et al. (1996) The CER3 gene of Arabidopsis thaliana is expressed in leaves, stems, roots, flowers and apical meristems. Plant J, 10: 459-67).

Of course, a variety of other structural genes of interest that can be operably linked to the regulatory sequence of the invention are available. For example, herbicides such as a mutated 5-enolpyruvyl-2-phosphoshikimate synthase can be expressed to provide decreased sensitivity to glyphosate. Such sequences can also provide for the expression of a gene product involved in detoxification of bromoxynil. Sequences may also be utilized relating to enhanced resistance to stress (such as provided by a gene for superoxide dismutase), temperature changes, osmotic pressure changes, and salinity (such as a gene associated with the overproduction of proline) and the like. Antisense sequences can be used to reduce other phenotypic traits.

In another embodiment, the invention provides a method for producing a genetically modified plant characterized as having an increased expression of a particular gene of interest expressed in the L1 layer of the epidermis of the plant as compared to a plant which has not been genetically modified (e.g., a wild-type plant). The method includes the steps of contacting a plant cell with at least one vector containing at least one nucleic acid sequence encoding a gene of interest, wherein the nucleic acid sequence is operably associated with an ESRS, to obtain a transformed plant cell; producing a plant from the transformed plant cell; and thereafter selecting a plant exhibiting expression of the gene of interest in the epidermis of the plant.

The term “genetic modification” as used herein refers to the introduction of one or more heterologous nucleic acid sequences into one or more plant cells, which can generate whole, sexually competent, viable plants. The term “genetically modified” as used herein refers to a plant which ahs been generated through the aforementioned process. Genetically modified plants of the present invention are capable of self-pollinating or cross-pollinating with other plants of the same species so that the foreign gene, carried in the germ line, can be inserted into or bred into agriculturally useful plant varieties. The term “plant cell” as used herein refers to protoplasts, gamete producing cells, and cells which regenerate into whole plants. Accordingly, a seed comprising multiple plant cells capable of regenerating into a whole plant, is included in the definition of “plant cell.”

As used herein, the term “plant” refers to either a whole plant, a plant part, a plant cell, or a group of plant cells, such as plant tissue, for example. Plantlets are also encompassed within the meaning of “plant”. Plants included in the invention are any plants amenable to transformation techniques, including, without limitation, angiosperms, gymnosperms, monocotyledons, and dicotyledons.

Examples of monocotyledonous plants include, but are not limited to, asparagus, field and sweet corn, barley, wheat, rice, sorghum, onion, pearl millet, rye and oats. Examples of dicotyledonous plants include, but are not limited to tomato, tobacco, cotton, rapeseed, field beans, soybeans, peppers, lettuce, peas, alfalfa, clover, cole crops or Brassica oleracea (e.g., cabbage, broccoli, cauliflower, brussel sprouts), radish, carrot, beets, eggplant, spinach, cucumber, squash, melons, cantaloupe, sunflowers and various ornamentals. Woody species include poplar, pine, sequoia, cedar, oak, etc.

Genetically modified plants are produced by contacting a plant cell with a vector including at least one nucleic acid sequence encoding a gene of interest operably associated with the ESRS. The ESRS is effective in the plant cells to cause transcription of gene of interest. In some embodiments, the ESRS is a ML1 regulatory sequence. Additionally, a polyadenylation sequence or transcription control sequence, also recognized in plant cells may also be employed. It is preferred that the vector harboring the nucleic acid sequence to be inserted also contain one or more selectable marker genes so that the transformed cells can be selected from non-transformed cells in culture, as described herein.

Optionally, a selectable marker may be associated with the nucleic acid sequence to be inserted. As used herein, the term “marker” refers to a gene encoding a trait or phenotype which permits the selection of, or the screening for, a plant or plant cell containing the marker. Preferably, the marker gene is an antibiotic resistance gene whereby the appropriate antibiotic can be used to select for transformed cells from among cells that are not transformed. Examples of suitable selectable markers include adenosine deaminase, dihydrofolate reductase, hygromycin-B-phospho-transferase, thymidine kinase, xanthine-guanine phospho-ribosyltransferase and amino-glycoside 3′-O-phospho-transferase II (kanamycin, neomycin and G418 resistance). Other suitable markers will be known to those of skill in the art.

Vector(s) employed in the present invention for transformation of a plant cell include a nucleic acid sequence encoding a gene of interest operably associated with the ESRS. To commence a transformation process in accordance with the present invention, it is first necessary to construct a suitable vector and properly introduce it into the plant cell. Details of the construction of vectors utilized herein are known to those skilled in the art of plant genetic engineering.

Nucleic acid sequences utilized in the present invention can be introduced into plant cells using Ti plasmids of Agrobacterium tumefaciens, root-inducing (Ri) plasmids, and plant virus vectors. (For reviews of such techniques see, for example, Weissbach & Weissbach, 1988, Methods for Plant Molecular Biology, Academic Press, NY, Section VIII, pp. 421-463; and Grierson & Corey, 1988, Plant Molecular Biology, 2d Ed., Blackie, London, Ch. 7-9, and Horsch, et al., Science, 227:1229, 1985, both incorporated herein by reference). In addition to plant transformation vectors derived from the Ti or root-inducing (Ri) plasmids of Agrobacterium, alternative methods may involve, for example, the use of liposomes, electroporation, chemicals that increase free DNA uptake, transformation using viruses or pollen and the use of microprojection.

One of skill in the art will be able to select an appropriate vector for introducing the gene of interest-encoding nucleic acid sequence in a relatively intact state. Thus, any vector which will produce a plant carrying the introduced DNA sequence should be sufficient. Even use of a naked piece of DNA would be expected to confer the properties of this invention, though at low efficiency. The selection of the vector, or whether to use a vector, is typically guided by the method of transformation selected.

The transformation of plants in accordance with the invention may be carried out in essentially any of the various ways known to those skilled in the art of plant molecular biology. (See, for example, Methods of Enzymology, Vol. 153, 1987, Wu and Grossman, Eds., Academic Press, incorporated herein by reference). As used herein, the term “transformation” means alteration of the genotype of a host plant by the introduction of a nucleic acid sequence with the ESRS.

For example, a nucleic acid sequence of interest can be introduced into a plant cell utilizing Agrobacterium tumefaciens containing the Ti plasmid, as mentioned briefly above. In using an A. tumefaciens culture as a transformation vehicle, it is most advantageous to use a non-oncogenic strain of Agrobacterium as the vector carrier so that normal non-oncogenic differentiation of the transformed tissues is possible. It is also preferred that the Agrobacterium harbor a binary Ti plasmid system. Such a binary system comprises 1) a first Ti plasmid having a virulence region essential for the introduction of transfer DNA (T-DNA) into plants, and 2) a chimeric plasmid. The latter contains at least one border region of the T-DNA region of a wild-type Ti plasmid flanking the nucleic acid to be transferred. Binary Ti plasmid systems have been shown effective to transform plant cells (De Framond, Biotechnology, 1: 262, 1983; Hoekema, et al., Nature, 303:179, 1983). Such a binary system is preferred because it does not require integration into the Ti plasmid of Agrobacterium, which is an older methodology.

Methods involving the use of Agrobacterium in transformation according to the present invention include, but are not limited to: 1) co-cultivation of Agrobacterium with cultured isolated protoplasts; 2) transformation of plant cells or tissues with Agrobacterium; or 3) transformation of seeds, apices or meristems with Agrobacterium. In addition, gene transfer can be accomplished by in planta transformation by Agrobacterium, as described by Bechtold, et al., (C. R. Acad. Sci. Paris, 316:1194, 1993) and exemplified in the Examples herein. This approach is based on the vacuum infiltration of a suspension of Agrobacterium cells.

One method of introducing a nucleic acid encoding a product of interest and operably associated with an ESRS into plant cells is to infect such plant cells, an explant, a meristem, or a seed, with transformed Agrobacterium tumefaciens as described above. Under appropriate conditions known in the art, the transformed plant cells are grown to form shoots, roots, and develop further into plants.

Alternatively, the nucleic acid sequences encoding a product of interest and operably linked to an ESRS can be introduced into a plant cell using mechanical or chemical means. For example, the nucleic acid can be mechanically transferred into the plant cell by microinjection using a micropipette. Alternatively, the nucleic acid may be transferred into the plant cell by using polyethylene glycol which forms a precipitation complex with genetic material that is taken up by the cell.

The ESRS linked to nucleic acid sequences encoding a product of interest can also be introduced into plant cells by electroporation (Fromm, et al., Proc. Natl. Acad. Sci., U.S.A., 82:5824, 1985, which is incorporated herein by reference). In this technique, plant protoplasts are electroporated in the presence of vectors or nucleic acids containing the relevant nucleic acid sequences. Electrical impulses of high field strength reversibly permeabilize membranes allowing the introduction of nucleic acids. Electroporated plant protoplasts reform the cell wall, divide and form a plant callus. Selection of the transformed plant cells with the transformed gene can be accomplished using phenotypic markers as described herein.

Another method for introducing an ESRS operably linked to nucleic acid encoding for a gene of interest into a plant cell is high velocity ballistic penetration by small particles with the nucleic acid to be introduced contained either within the matrix of such particles, or on the surface thereof (Klein, et al., Nature 327:70, 1987). Bombardment transformation methods are also described in Sanford, et al. (Techniques 3:3-16, 1991) and Klein, et al. (Bio/Techniques 10:286, 1992). Although, typically only a single introduction of a new nucleic acid sequence is required, this method particularly provides for multiple introductions.

Cauliflower mosaic virus (CaMV) may also be used as a vector for introducing the nucleic acid sequence plus ESRS into plant cells (U.S. Pat. No. 4,407,956). CaMV viral DNA genome is inserted into a parent bacterial plasmid creating a recombinant DNA molecule which can be propagated in bacteria. After cloning, the recombinant plasmid again may be cloned and further modified by introduction of the desired nucleic acid sequence. The modified viral portion of the recombinant plasmid is then excised from the parent bacterial plasmid, and used to inoculate the plant cells or plants.

In another embodiment, the invention affords a method of providing increased transcription of a nucleic acid sequence expressing a product of interest in the Li layer of the epidermis of a plant. The method comprises providing a plant having integrated into its genome a nucleic acid sequence encoding the protein of interest construct. As used herein, the term “contacting” refers to any means of introducing the regulatory sequence operably linked to the nucleic acid sequence encoding for a gene of interest into the plant cell, including chemical and physical means as described above. Preferably, contacting refers to introducing the nucleic acid or vector into plant cells (including an explant, a meristem or a seed), via Agrobacterium tumefaciens transformed with the gene encoding nucleic acid and regulatory sequence as described above.

Normally, a plant cell is regenerated to obtain a whole plant from the transformation process. The immediate product of the transformation is referred to as a “transgenote”. The term “growing” or “regeneration” as used herein means growing a whole plant from a plant cell, a group of plant cells, a plant part (including seeds), or a plant piece (e.g., from a protoplast, callus, or tissue part).

Regeneration from protoplasts varies from species to species of plants, but generally a suspension of protoplasts is first made. In certain species, embryo formation can then be induced from the protoplast suspension, to the stage of ripening and germination as natural embryos. The culture media will generally contain various amino acids and hormones, necessary for growth and regeneration. Examples of hormones utilized include auxins and cytokinins. It is sometimes advantageous to add glutamic acid and proline to the medium, especially for plant species such as corn and alfalfa. Efficient regeneration will depend on the medium, on the genotype, and on the history of the culture. If these variables are controlled, regeneration is reproducible.

Regeneration also occurs from plant callus, explants, organs or parts. Transformation can be performed in the context of organ or plant part regeneration. (see Methods in Enzymology, Vol. 118 and Klee, et al., Annual Review of Plant Physiology, 38:467, 1987). Utilizing the leaf disk-transformation-regeneration method of Horsch, et al., Science, 227:1229, 1985, disks are cultured on selective media, followed by shoot formation in about 2-4 weeks. Shoots that develop are excised from calli and transplanted to appropriate root-inducing selective medium. Rooted plantlets are transplanted to soil as soon as possible after roots appear. The plantlets can be repotted as required, until reaching maturity.

In vegetatively propagated crops, the mature transgenic plants are propagated by utilizing cuttings or tissue culture techniques to produce multiple identical plants. Selection of desirable transgenotes is made and new varieties are obtained and propagated vegetatively for commercial use.

In seed propagated crops, the mature transgenic plants can be self crossed to produce a homozygous inbred plant. The resulting inbred plant produces seed containing the newly introduced foreign gene(s). These seeds can be grown to produce plants that would produce the selected phenotype, e.g. increased yield.

Parts obtained from regenerated plant, such as flowers, seeds, leaves, branches, roots, fruit, and the like are included in the invention, provided that these parts comprise cells that have been transformed as described. Progeny and variants, and mutants of the regenerated plants are also included within the scope of the invention, provided that these parts comprise the introduced nucleic acid sequences.

Plants exhibiting increased expression of a particular phenotype as compared with wild-type plants can be selected by visual observation. The invention includes plants produced by the method of the invention, as well as plant tissue, seeds, and other plant cells derived from the genetically modified plant.

The above disclosure generally describes the various embodiments. A more complete understanding can be obtained by reference to the following specific examples which are provided herein for purposes of illustration only and are not intended to limit the scope of the invention.

### EXAMPLES

Isolation of the ML1 coding and noncoding nucleic acid sequences was performed. To test whether the ML1 regulatory sequence is sufficient to drive expression of a heterologous gene, the regulatory sequence was fused to the structural gene β-glucuronidase (GUS). Transgenic Arabidopsis and tobacco plants that carry a fusion of the ML1 regulatory sequence to a reporter gene encoding β-glucuronidase (GUS) were constructed. These plants expressed high levels of GUS in the L1 layer of the epidermis of meristems, as determined by histochemical staining with the GUS substrate X-gluc (5-bromo-4-chloro-3-indoyl β-D-glucuronide).

The ATML1 regulatory sequences described herein are useful for a variety of transgenic experiments. For example, several genes that control meristem and flower development have been suggested to act non-autonomously (Clark et al., Cell, 89: 575-585, 1997; Mayer et al., Cell 8, 805-815, 1998; Fletcher et al., Science 283, 1911-1914, 1999) and their layer autonomy can be tested using the ATML1 regulatory sequence. The ATML1::GUS reporters on their own should also provide a convenient marker for L1 identity in embryonic mutants that have defects in the layer organization (Mayer et al., Nature 353, 402-407, 1991).

### Example 1

### Identification of Epidermis-specific Transcriptional Regulatory Elements

Alignment of ATML1 cDNA (Lu et al., 1996) (SEQ ID NO: 1) and genomic sequences suggests that the ATML1 transcription unit comprises 11 exons. (Note: the ATML1 cDNA sequence is represented in SEQ ID NO: 1 and the protein corresponding to ATML1 cDNA sequence is detailed in SEQ ID NO: 2). The 5′ region of ATML1 is shown as a diagram in FIG. 1. The transcription start is tentatively defined as the 5′ end of the longest cDNA (Lu et al., 1996) (SEQ ID NO: 1). The alignment shows that transcription starts at least 1.8 kb upstream of the predicted first ATG, which lies in the third exon. The entire ATML1 genomic region has been sequenced (GenBank Accession #AL035527).

ATML1 Genomic Clones

A 551 base pair fragment, generated by reverse transcription coupled to polymerase chain reaction (RT-PCR) and corresponding to nucleotides 926-1476 of the ATML1 cDNA (Lu et al., 1996), is shown as SEQ ID NO: 4. Using this fragment, three overlapping genomic clones were isolated from an Arabidopsis genomic library (λAS1, λAS2, and λAS6).

Reporter Constructs

Coordinates are relative to the start of the first exon (Lu et al., 1996) (Genbank AL035527).

ML1::GUS.1: A 3.5 kb Xba I fragment from λAS2 extending from −200 region to the beginning of exon 3 was subcloned into pBluescriptSK+ (Stratagene, San Diego, Calif.) to create pAS85. A fragment extending from −200 to just upstream of the predicted first ATG was amplified from pAS85 by PCR with primer 83 (AAAAAGCTTAGTCTCGAAATCCTTC) (SEQ ID NO: 5) and a T7 primer, cut with Hind III, and cloned into pBluescriptSK+ and pBI101.1 (Jefferson et al., Proc. Natl. Acad. Sic. USA 83, 8447-8451, 1985) to create pAS95 and pAS92 (ML1::GUS.1), respectively.

ML1::GUS.2: A 4.4 kb BamH I fragment from λAS6 extending from −4 kb to just downstream of exon 1 was subcloned into pBluescriptSK+ to create pAS76. A fragment extending from −3.5 kb upstream of the beginning of exon 1 was amplified from pAS76 by PCR using primer 87 (TTTAAGCTTAACCGGTGGATTCAGGG) (SEQ ID NO: 6) and a T7 primer, cut with Hind III, and cloned into pBI10.1 to create pAS103 (ML1::GUS.2).

ML1::GUS.3: A fragment extending from −3 kb to just upstream of the predicted first ATG was cloned into the Xba I site of pBluescriptSK+ using a three-way cloning involving the 3.0 kb Xba I fragment of pAS76, and the 2.0 kb Xba I fragment of pAS95, creating pAS106. The 5.8 kb Hind III fragment of pAS106 was inserted into pBI101.1 to create pASIIO (MLJ::GUS.3).

Constructs were introduced into the Columbia (Col-0) ecotype using Agrobacterium-mediated vacuum transformation (Bechtold et al., C. R. Acad. Sci. 316, 1194-1199, 1993). Transformants were selected on MS plates containing kanamycin.

To identify epidermis-specific transcriptional regulatory elements, three transformation vectors were made in which the GUS reporter gene (Jefferson et al., 1985) was transcriptionally fused to ATML1 regulatory sequences (FIG. 1). The ML1::GUS 1 construct contained 200 bp of regulatory sequences along with the first two exons and introns and part of the third exon, ML1::GUS 2 contained 3.5 kb of regulatory sequence and part of the first exon, and ML1::GUS 3 contained 3 kb of regulatory sequence along with the first two exons and introns and part of the third exon (FIG. 1). Except for the 5′ most 0.5 kb, ML1::GUS 3 combined the ATML1 sequences present in ML1::GUS 1 and ML1::GUS 2, which overlap for about 200 bp.

### Example 2

### Analysis of GUS mRNA in T2 Siblings

T2 siblings of four T1 lines of each construct in the Col-0 background were analyzed for accumulation of GUS mRNA in their inflorescence meristems and young floral buds using in situ hybridization. Table 1(a) gives the distribution of expression patterns found in individual ML1::GUS1, ML1::GUS2, and ML1::GUS3 lines.

TABLE 1
Distribution of staining patterns conferred by the three
ML1::GUS constructs as assayed by (a) GUS RNA
accumulation and (b) GUS activity. Class I, II, III
and IV are the general staining patterns as
described in the text.

| | | | Class | | | | |
|---|---|---|---|---|---|---|---|
| | Construct | n^{a} | I | Class II | Class III | Class IV | None |
| (a) | ML1::GUS1 | 4 | — | 25% | — | — | 75% |
| | ML1::GUS2 | 4 | 50% | 25% | 25% | — | — |
| | ML1::GUS3 | 4 | 25% | — | 50% | — | 25% |
| (b) | ML1::GUS1 | 11 | 9% | — | — | 9% | 82% |
| | ML1::GUS2 | 14 | 36% | 7% | 43% | 14% | — |
| | ML1::GUS3 | 10 | 10% | 10% | 70% | 10% | — |

^{a}Number of lines scored. In experiment (a), four T2 individuals per line were assayed. In experiment (b), 10 T2 individuals were stained and at least four of these 10 were sectioned.

Lines from each construct expressed GUS mRNA specifically within the L1 layer, but each line varied in the relative amount and timing of expression in the inflorescence and floral meristems. Four general classes of temporal expression patterns were observed.

Only strong lines showed expression in the inflorescence meristem. In addition, strong lines showed expression during young stages of flower development. Intermediate lines showed expression in floral primordia from stage 1 on. Moderate lines showed expression after stage 1 of flower development. Weak lines showed expression after stage 5 of flower development. These results suggest that the ML1 ESRS regulatory sequence activity increases from the meristem stage through stage 5 of flower development.

Strong epidermis-specific transcriptional regulatory elements were found in the regulatory sequence-proximal region of ATML1. The overlap of the three reporter constructs tested here includes a 200 base pair region that is located just upstream of the presumed transcription start and that might contain either an L1-specific enhancer, or an L2/L3-specific silencer. Redundant elements might lie in the −3.5 kb region and in the first or second intron.

### Example 3

### Analysis of Relative Strengths of Regulatory Sequence Fragments

To compare the relative strengths of the three ML1 regulatory sequence fragments, T2 siblings of 10 lines of each construct were analyzed for GUS activity in their inflorescence meristems and young floral buds.

GUS Assays

Tissue was prefixed in ice cold 90% acetone for 20 minutes on ice, rinsed with cold water for 5 minutes, vacuum infiltrated for 5 minutes on ice with staining solution (50 mM sodium phosphate buffer pH 7.0, 0.2% Triton-X-100, 10 mM potassium ferrocyanide, 10 mM potassium ferricyanide, 1 mM X-gluc) and incubated at 37° C. for 12 hours. Samples were changed through 30-minute steps of 20% ethanol, 30% ethanol, 50% ethanol, FAA (50% ethanol, 5% acetic acid, 3.7% formaldehyde), dehydrated through an ethanol series into Histoclear (National Diagnostics), and embedded in Paraplast Plus. 8 μm sections were viewed after deparaffinization under Nomarski optics.

In order to compare inflorescence staining among the three ML1::GUS constructs, for each construct the emerging inflorescence shoots of at least 10 T2 siblings from at least 10 lines were stained for GUS activity. Four to 8 stained individuals from each line that showed GUS activity were embedded and sectioned (109 total) and the staining pattern of the majority of individuals recorded. A similar strategy was used to compare staining of 14-day old seedlings among 12 independent ML1::GUS.2 lines (62 individuals sectioned).

In situ hybridization was performed according to C. Ferrándiz, Q. Gu, R. Martienssen, M. F. Yanofsky, Development 127, 725-734 (2000). Digoxigenin labeled GUS antisense RNA probes were generated according manufacturers specifications (Boehringer Mannheim) using pLS27 as a template (Blázquez et al., Development 124, 3835-3844, 1997).

Results

All lines showed GUS activity in the epidermis, but varied in the level and the onset of expression in the shoot apex. Table 1(b) summarizes the staining patterns found in the majority of siblings in each independent line. In general, ML1::GUS 2 lines had higher levels of GUS mRNA and enzyme activity than either ML1::GUS 1 or ML1::GUS 3 lines.

Low levels of GUS staining were often observed in subepidermal layers of lines with the highest levels of expression. Staining in L2 and L3 increased when the concentration of ferri- and ferrocyanide salts in the GUS assay buffer was below 10 mM. To determine whether the subepidermal staining reflected low levels of GUS RNA expression, parallel experiments were conducted in which siblings from individual lines were either stained for GUS activity in varying concentrations of ferri- and ferrocyanide, or assayed for GUS RNA expression. These experiments showed that GUS RNA expression was always restricted to the epidermis, while only GUS staining was also detected in subepidermal layers in a ferri- and ferrocyanide dependent manner. Potassium ferri- and ferrocyanide concentrations above 10 mM caused a decrease in GUS activity. It appears that subepidermal staining is either due to leakage of GUS enzyme or of the X-gluc reaction product.

### Example 4

### Analysis of Epidermis-specific Expression During Arabidopsis Life Cycle

To explore whether the 3.5 kb fragment could direct epidermis-specific expression during other stages of the Arabidopsis life cycle, ten independent ML1::GUS2 lines were stained for GUS RNA or protein activity in embryos and 14-day old plants undergoing the transition to reproductive development. In general, lines with strong L1-specific expression in the inflorescence shoot apex also showed high levels of L1-specific expression in the shoot meristems undergoing the transition from vegetative to reproductive development. Intermediate lines, which showed only expression in floral, but not shoot meristems of reproductive apices, also lacked L1 expression in the transition meristem. Epidermis-specific GUS RNA expression in embryos was only observed with strong lines.

Unexpectedly, GUS activity was also detected in the tips of the primary and lateral roots in most ML1::GUS 2 lines examined. However, this activity was confined to the epidermis of the root cap and meristem and to the L1 of initiating lateral roots.

### Example 5

### Transformation of Tobacco Plants with ML1 Regulatory Sequence and GUS Reporter

Employing the methods described with reference to Examples 1-4, constructs including ML1:GUS 2 and ML1:GUS 3 were introduced into tobacco plants, using Agrobacterium-mediated transformation of tobacco leaf disks (R. B. Horsch et al., Science 227, 1229-1231 (1985)). Table 2 details the distribution of staining patterns conferred by the two ML1:GUS constructs.

TABLE 2
ML1::GUS Functions in Tobacco

| | | Distribution of staining patterns conferred by | | | |
|---|---|---|---|---|---|
| | | the two ML1::GUS constructs assayed | | | |
| Construct | n | Strong | Intermediate | Weak | None |
| ML1::GUS2 | 17 | 3 | 1 | 1 | 12 |
| ML1::GUS3 | 52 | 4 | 5 | 6 | 37 |

^{a} Number of lines scored.

Lines from each construct expressed GUS mRNA specifically within the L1 layer of the epidermis of the tobacco plants. These results suggest that the ML1 ESRS is effective in generating epidermal-specific expression of the GUS reporter in the epidermis of a tobacco plant, as well as an Arabidopsis thaliana plant.

The foregoing description details certain embodiments of the invention. It will be appreciated, however, that no matter how detailed the foregoing appears in text, the invention can be practiced in many ways. As is also stated above, it should be noted that the use of particular terminology when describing certain features or aspects of the invention should not be taken to imply that the terminology is being re-defined herein to be restricted to including any specific characteristics of the features or aspects of the invention with which that terminology is associated. The scope of the invention should therefore be construed in accordance with the appended claims and any equivalents thereof.

What is claimed is:

1. An isolated epidermal specific regulatory nucleic acid, wherein operably associating said regulatory nucleic acid with a heterologous nucleic acid results in epidermal specific expression of said heterologous nucleic acid.

2. The isolated epidermal specific regulatory nucleic acid of claim 1, wherein said epidermal specific regulatory nucleic acid is derived from an ATML1 gene.

3. The isolated epidermal specific regulatory nucleic acid of claim 1, wherein said epidermal specific regulatory nucleic acid is a ML1 epidermal specific regulatory nucleic acid.

4. The isolated epidermal specific regulatory nucleic acid of claim 3, wherein said ML1 epidermal specific regulatory nucleic acid comprises the sequence of SEQ ID NO: 3, or functional fragments thereof.

5. The isolated epidermal specific regulatory nucleic acid of claim 1, wherein said heterologous nucleic acid encodes a Bacillus thuringiensis toxin.

6. A transgenic plant cell comprising an epidermal specific regulatory nucleic acid operably associated with a heterologous nucleic acid, wherein a plant derived from said plant cell specifically expresses said heterologous nucleic acid in the epidermis.

7. The transgenic plant cell of claim 6, wherein said plant cell is a monocotyledonous or dicototyledonous plant cell.

8. The transgenic plant cell of claim 6, wherein said epidermal specific regulatory nucleic acid is derived from an ATML1 gene

9. The transgenic plant cell of claim 6, wherein said epidermal specific regulatory nucleic acid is a ML1 epidermal specific regulatory nucleic acid.

10. The transgenic plant cell of claim 6, wherein said ML1 epidermal specific regulatory nucleic acid comprises the sequence of SEQ ID NO: 3 or functional fragments thereof.

11. The transgenic plant cell of claim 6, wherein said heterologous nucleic acid encodes a Bacillus thuringiensis toxin.

12. The transgenic plant cell of claim 6, wherein said transgenic plant cell is a tobacco plant cell.

13. The transgenic plant cell of claim 6, wherein said transgenic plant cell is an Arabidopsis thaliana plant cell.

14. The transgenic plant cell of claim 6, further comprising a selectable marker.

15. A transgenic plant comprising an epidermal specific regulatory nucleic acid operably associated with a heterologous nucleic acid, wherein said heterologous nucleic acid is specifically expressed in the epidermal layer of the plant.

16. The transgenic plant of claim 15, wherein said plant is a monocotyledonous or a dicotyledonous plant.

17. The transgenic plant of claim 15, wherein said plant is Arabidopsis thaliana.

18. The transgenic plant of claim 15, wherein said plant is a tobacco plant.

19. The transgenic plant of claim 15, further comprising a selectable marker.

20. The transgenic plant of claim 15, wherein said epidermal specific regulatory nucleic acid is derived from an ATML1 gene.

21. The transgenic plant of claim 15, wherein said epidermal specific regulatory nucleic acid is a ML1 epidermal specific regulatory nucleic acid.

22. The transgenic plant of claim 21, wherein said ML1 epidermal specific regulatory nucleic acid comprises the sequence of SEQ ID NO: 3, or functional fragments thereof.

23. The transgenic plant of claim 15, wherein said heterologous nucleic acid encodes a Bacillus thuringiensis toxin.

24. An expression vector comprising an epidermal specific regulatory nucleic acid operably associated with a heterologous nucleic acid.

25. The expression vector of claim 24, wherein said expression vector is selected from the group consisting of a retroviral vector, a Ti plasmid, and Cauliflower mosaic virus (CaMV).

26. The expression vector of claim 24, wherein said epidermal specific regulatory nucleic acid is derived from an ATML1 gene.

27. The expression vector of claim 24, wherein said epidermal specific regulatory nucleic acid is a ML1 epidermal specific regulatory nucleic acid.

28. The expression vector of claim 24, wherein said ML1 epidermal specific regulatory nucleic acid comprises the sequence of SEQ ID NO: 3, or functional fragments thereof.

29. The expression vector of claim 24, wherein said heterlogous nucleic acid encodes a Bacillus thuringiensis toxin.

30. A method of specifically expressing a heterologous nucleic acid in the epidermis of a plant comprising:
transforming a plant with a nucleic acid construct comprising an epidermal specific regulatory nucleic acid operably associated with said heterologous nucleic acid; and
selecting for plants that exhibit epidermal specific expression of said heterologous nucleic acid.

31. The method of claim 30, wherein said transforming a plant comprises transforming an Arabidopsis thaliana plant.

32. The method of claim 30, wherein said transforming a plant comprises transforming a tobacco plant.

33. The method of claim 30, wherein said heterologous nucleic acid encodes a Bacillus thuringiensis toxin.
